(19) United States
(12) Patent Application Publication
(10) Pub. No.: US 2004/0067262 A1
(43) Pub. Date: Apr. 8, 2004

(54) Methods and devices for treating diffuse exostosis and related conditions

(76) Inventor: Brent R. Constantz, Cupertino, CA (US)

Correspondence Address: OnFlow Corporation, c/o Joseph Waechter, 32 Fallen Leaf Terrace, Orinda, CA, 94563, US

(21) Appl. No.: 10/265,286

(22) Filed: Oct. 4, 2002

(51) Int. Cl. A61K 33/18

(52) U.S. Cl. 424/666

(57) ABSTRACT

Methods for at least inhibiting the growth of an inorganic structure in an external auditory canal are provided. In practicing the subject methods, the target structure is contacted with an amount of an inorganic matter dissolution solution effective to at least inhibit inorganic structure growth, e.g., by flushing the external auditory canal with an effective amount of an inorganic matter dissolution solution. In addition, the invention provides devices, systems and kits for practicing the subject methods. The subject invention finds use in the treatment of conditions characterized by inorganic growths in the external auditory canal, including diffuse exostosis, tympanic sclerosis, and the like.

### Technical Field

The field of this invention is the treatment of conditions associated with inorganic structures in the external auditory canal, e.g., diffuse exostosis and related conditions.

### BACKGROUND OF THE INVENTION

Diffuse exostosis is a condition that is prevalent among those who frequently expose their ears to water for long periods of time. Under normal conditions, the cerumen (or ear wax) acts to waterproof the external auditory canal, and any moisture present therein is evaporated. However, chronic exposure to moisture in the ear canal overwhelms the body's natural defenses, and diffuse exostosis, or possibly tympanic sclerosis (thickening of the eardrum), is the end result. Diffuse exostosis is characterized by the progressive build-up of inorganic structures in the bony medial external auditory canal. These non-uniform growths prevent the normal cleaning mechanisms of the ear to function, such that bacteria, excess moisture, cerumen, dead skin cells, etc., are not removed and eventually cause occlusion of the ear canal.

If left untreated, diffuse exostosis results in hearing impairment or loss. In addition, the risk of infection is increased, and if infected, the swelling can cause chronic pain to the individual. Retained water around the debris can cause maceration of the ear canal skin and increases the likelihood of infection. Even a mild amount of swelling of the ear canal skin in response to acute infection may cause complete obstruction of the external auditory canal.

The best treatment for diffuse exostosis is prevention. The use of earplugs is frequently employed by those who expose their ears to cold environments, e.g., surfers, kayakers, etc. Current methods used to manage an existing diffuse exostosis include application of a solution of white vinegar and alcohol (e.g., a mixture of one ounce of vinegar and one quart of alcohol), which may be used to remove bacteria and fungal growth from the ear canal. Another method used to manage diffuse exostosis is to apply an alcohol lavage (91% isopropyl alcohol), which may be flushed into the ear. These procedures do nothing to remove the bony growths from the ear canal, they merely prevent the existing situation from worsening.

In cases of severe diffuse exostosis, where there is hearing loss, surgery may be required to remove the bony growths. Surgical procedures are costly, healing is slow and uncertain, and the bony growths may ultimately grow back.

Therefore, there is an interest in development of non-surgical treatments for diffuse exostosis that will do more than manage the existing condition. The present invention satisfies this need.

### SUMMARY OF THE INVENTION

Methods are provided for at least inhibiting the growth of inorganic structures in the external auditory canal. In practicing the subject methods, an inorganic matter dissolution solution is contacted with the target inorganic structures, e.g., by flushing the external auditory canal with the inorganic matter dissolution solution, for a period of time sufficient to at least inhibit growth of the target inorganic structures. Also provided are devices, systems and kits for use in practicing the subject methods. The subject invention finds use in the treatment of conditions characterized by the presence of inorganic growths/structures in the external auditory canal, including diffuse exostosis, tympanic sclerosis, and the like.

### BRIEF DESCRIPTION OF THE FIGURES

FIGS. 1A and 1B provide photographic and high resolution contact radiograph of human tympanic membrane sample, respectively. The high resolution contact radiograph confirms the presence of mineral within the sample.

FIG. 2. The normalized mineral dissolved as a function of time for a calcific human tympanic membrane exposed to 0.1N CDS solution.

### DESCRIPTION OF THE SPECIFIC EMBODIMENTS

Methods are provided for at least inhibiting the growth of an inorganic structure in an external auditory canal. In practicing the subject methods, the target inorganic structure (s) is contacted with an inorganic matter dissolution solution, e.g., by flushing the external auditory canal with an effective amount of an inorganic matter dissolution solution, to achieve the desired inhibition in inorganic structure growth. The subject methods find use in the treatment of conditions characterized by the presence of inorganic/bony growths in the external auditory canal, including diffuse exostosis, tympanic sclerosis, and the like. Also provided are devices, systems and kits for use in practicing the subject methods. In further describing the subject invention, the subject methods are discussed first in greater detail, followed by a review of the subject devices, systems and kits.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

It must be noted that, as used in this specification and the appended claims, the singular forms “a,” “an” and “the” include plural reference unless the context clearly dictates otherwise. Unless defined otherwise all technical and scientific terms used herein have the same meaning as commonly understood to one of ordinary skill in the art to which this invention belongs.

Where a range of values is provided, it is understood that each intervening value, to the tenth of the unit of the lower limit, unless the context clearly dictates otherwise, between the upper and lower limit of that range and any other stated or intervening value in that stated range, is encompassed within the invention. The upper and lower limits of these smaller ranges may independently be included in the smaller ranges, and such embodiments are also encompassed within the invention, subject to any specifically excluded limit in the stated range. Where the stated range includes one or both of the limits, ranges excluding either or both of those included limits are also included in the invention.

All publications mentioned herein are incorporated herein by reference for the purpose of describing and disclosing components that are described in the publications that might be used in connection with the presently described invention.

Methods

As summarized above, the subject invention provides methods of at least inhibiting the growth of inorganic structures in the external auditory canal of a host. By at least inhibiting the growth of inorganic structures is meant at least reducing the rate at which the target inorganic structure(s) of the canal increases in mass, e.g., grows, where in many embodiments, the rate of growth is at least slowed by a factor of at least about 1.5 fold, usually by at least about 5 fold and more usually by at least about 10 fold, where in many embodiments the decrease in rate of growth may be as great as about 25 fold, 50 fold, 75 fold or greater, as compared to a control, e.g., the rate of growth of an inorganic structure not contacted with a dissolution solution according to the subject methods. In certain embodiments, at least inhibiting growth means the prevention of any further growth of the target inorganic structure(s) from occurring in the external auditory canal, such that the inorganic structure may not decrease in size, but does not substantially increase is size either, where any increase in size is less than about 5%, usually less than about 3% and more usually less than about 1% by weight. In many embodiments, the subject methods result in a reduction in size of the target inorganic structures, where the magnitude of the reduction is generally at least about 5%, usually at least about 10% and more usually at least about 25% by weight, where in many embodiments the magnitude of the reduction may be as great as 50%, 75% or greater, up to and including complete removal of the target inorganic structure(s) from the external auditory canal.

The target inorganic structures of the subject methods are non-carbon based structures that are present in the external auditory canal. The inorganic structures originate in the periosteum (covering the surface of the bone) of the external auditory canal and manifest as a concentric lamellar bony structure without any vascular tissue. The size of the inorganic structure may vary from so small that there is no ill effect on the host, all the way up to the diameter of the external auditory canal, causing a total occlusion of the canal. The maximum size of the inorganic structure will depend on how big the external auditory canal of the host is, how long the inorganic structure existed before treatment is sought, and whether any preventive measures have been taken. In many embodiments where the subject methods are indicated, the mass of the structure may be so great as to completely occlude the external auditory canal of the host. The target inorganic structure will commonly be a bony growth composed of at least some calcium-containing mineral component. Typically, the inorganic structures include to a large extent a calcium phosphate mineral component, e.g., hydroxyapatite, Ca₁₀(PO₄)₆(OH)₂, dahllite, etc.

As indicated above, the target inorganic structures are present in the external auditory canal of the host or subject being treated, where a single structure may be present or multiple structures may be present, where the structures may partially or complete occlude the auditory canal. The external auditory canal is well known to those of skill in the art, and is the open air passageway extending from the auricle to the tympanic membrane which is also referred to as the external auditory meatus. The external auditory canal is made up of a cartilaginous and osseous region, where in many embodiments the target inorganic structures are at least associated with, i.e., in the vicinity or region of, the osseous region.

In practicing the subject methods, following evaluation of the host external auditory canal and assessment that treatment according to the subject methods is indicated, the target inorganic structure is contacted with an inorganic matter dissolution solution, typically a solution of subphysiologic pH, for a period of time sufficient to achieve the desired inhibition of structure growth. As the solution employed in the subject methods is a solution of subphysiologic pH, it is also properly referred to as an acidic solution. A variety of different types of acidic solutions may be employed in the subject methods. The acidic treatment solutions that find use in the subject methods generally have a pH of less than about 6.5, where the pH is usually less than about 4.0 and more usually less than about 3.0. In many preferred embodiments, the pH ranges from 0 to 2, and usually 0 to 1.

The acidic treatment solution can include a number of different types of acids, where the acids may or may not include a hydrocarbon moiety, i.e., a hydrogen bonded directly to a carbon atom. Suitable acids that lack a hydrocarbon moiety include halogen acids, oxy acids and mixtures thereof, where specific acids of interest of this type include, but are not limited to, hydrochloric, nitric, sulfuric, phosphoric, hydroboric, hydrobromic, carbonic and hydroiotic acids. For such acids, the acid can be a concentrated acid, or can be diluted. Upon dilution, the concentration of an inorganic acid will generally be from about 10 N to about 0.01 N, preferably between 5 N to 0.1 N. Also of interest are acids that include a hydrocarbon moiety, where such acids include, but are not limited to, any organic acid of one to six (C₁ to C₆) carbons in length. Organic acids of this type include, but are not limited to, formic, acetic, propionic, maleic, butanoic, valeric, hexanoic, phenolic, cyclopentanecarboxylic, benzoic, and the like. For an organic acid, the acid can be in concentrated form, or can be diluted. The acidic treatment solution can be composed of either a monobasic or a polybasic acid. Acids are “monobasic” when they have only one replaceable hydrogen atom and yield only one series of salts (e.g., HCl). Acids are “polybasic” when they contain two or more hydrogen atoms which may be neutralized by alkalies and replaced by organic radicals.

In many embodiments of the subject invention, the acid solution is hypertonic, by which is meant that the osmolarity of the solution is greater than that of whole blood, i.e. the osomolarity is greater than 300 mosmol. The solution may be rendered hypertonic by including any convenient component or components in the solution which provide for the desired elevated osmolarity.

Any convenient agent that is capable of increasing the osmolarity of the solution may be employed, where suitable agents include salts, sugars, and the like. In many embodiments, the agent that is employed to render the solution hypertonic is one or more, usually no more than three, and more usually no more than two, different salts. Generally, the salt concentration in these embodiments of the solution is at least about 100 mosmol, usually at least about 200 mosmol and more usually at least about 300 mosmol, where the concentration may be as high as 3000 mosmol or higher, depending on the particular salt being employed to render the solution hypertonic, where the solution may be saturated with respect to the salt in certain embodiments. Salts that may be present in the subject solutions include: NaCl, MgCl₂, Ringers, etc. where NaCl is preferred in many embodiments.

Of particular interest in many embodiments is the use of a hydrogen chloride solution. In hydrogen chloride solutions that find use in the subject invention, the concentration of HCl in the solution ranges from about 0.001 to 1.0 N, usually from about 0.01 to 1.0 N and more usually from about 0.1 to 1.0 N. In many embodiments, the hydrogen chloride solution will further include one or more salts which make the solution hypertonic, as described above. In certain preferred embodiments, the salt is NaCl, where the concentration of NaCl in the solution is at least 0.05 M, usually at least 0.10 M, and more usually at least 0.15 M, where the concentration may be as high as 0.25 M or higher. In certain embodiments, the solution will be saturated with NaCl.

Of particular interest are aqueous hydrogen chloride solutions that consist of water, hydrogen chloride and NaCl. The concentration of hydrogen chloride in these solutions of particular interest ranges from about 0.01 to 1.0 N, usually from about 0.05 to 0.5 N and more usually from about 0.075 to 0.25 N. The concentration of NaCl in these solutions of particular interest ranges from about 0.05 to 0.25 M, usually from about 0.05 to 0.10 M.

In the subject methods, the target inorganic structure is contacted with the acidic solution for a period of time sufficient for the desire inhibition of growth, as described above, to be achieved. Contact with the target structure may be accomplished in any convenient manner, so long as it results in the desired growth inhibition.

The manner in which contact is achieved may be static or dynamic. By static is meant that a predetermined amount of acidic solution is introduced into the external auditory canal and maintained in the external auditory canal for the entire treatment period, without the addition of further quantities of acidic solution. By dynamic contact is meant that the fresh acidic solution is contacted with at the target structure one or more times, including continuously, during the treatment period.

In certain embodiments of the subject methods, the target structure(s) is continuously contacted or flushed with the acidic fluid. In other words, the acidic fluid is introduced in a manner such that a continuous flow of the acidic solution into the external auditory canal is achieved. Where the external auditory canal is flushed with the dissolution fluid, it is preferred that the pressure in the external auditory canal remains substantially isometric. By substantially isometric is meant that the pressure does not vary by a significant amount, where the amount of variance over the treatment period does not vary by more than about 50%, usually by not more than about 10% and more usually by not more than about 5%. In other words, the external auditory canal remains substantially isobaric during the treatment period. Accordingly, where fluid is dynamically contacted with the target structure, fluid is also simultaneously removed from the external auditory canal, such that the overall volume of fluid in the external auditory canal remains substantially constant, where any difference in volume at any two given times during the treatment period does not exceed about 50%, and usually does not exceed about 10%. As such, the dissolution fluid is introduced into the external auditory canal of the target lesion in a manner such that the external auditory canal remains substantially isovolumetric.

Where the acidic solution is dynamically introduced into and aspirated from the external auditory canal, the fluid is introduced in a manner such that the flow rate of the dissolution solution through the external auditory canal is generally at least about 10 cc/min, usually at least about 20 cc/min and more usually at least about 60 cc/min, where the flow rate may be as great as 120 cc/min or greater, but usually does not exceed about 1000 cc/minute and more usually does not exceed about 500 cc/minute, where by “volume” is meant the volume of the external auditory canal. The total amount of dissolution fluid that is passed through the external auditory canal during the treatment period typically ranges from about 100 to 1000 cc, usually from about 200 to 800 cc and more usually from about 400 to 500 cc. The solution may be pressurized to achieve the desired flow rate, as described supra. It is important to note that the overall pressure in the external auditory canal is maintained at substantially isometric or isobaric conditions.

The target structure is contacted, e.g. statically contacted or flushed (as described above), with the acidic treatment fluid or solution for a period of time sufficient for the desired growth inhibition to be achieved. Generally, the period of time during which the target structure is contacted with the acidic solution ranges from about 1 to 100 minutes, usually from about 1 to 30 minutes, where in certain embodiments contact is from about 1 to 5 minutes, in other embodiments contact is from about 5 to 10 minutes and in yet other embodiments contact is from about 10 to 30 minutes. Where contact is achieved by flushing the external auditory canal with the acidic solution, the contact duration typically lasts for a period of time ranging from about 5 to 30 minutes, usually from about 10 to 30 minutes and more usually from about 10 to 20 minutes.

In practicing the subject methods, the external auditory canal may be flushed a single time or multiple times over a given period of time. Thus, serial applications may take place, depending on the severity of the diffuse exostosis, the acidity of the solution applied, and the length of time the solution is allowed to remain in the external auditory canal. The applications of dissolution solution may be given over a period of days, weeks or months, depending on the circumstances of the individual patient.

Flushing the external auditory canal with the dissolution solution according to the methods described above results in at least growth inhibition of the target inorganic structures as described above.

In a number of embodiments, the method by which the bony growth is contacted by the dissolution solution may be modified to include a number of additional steps. Additional method steps that may be practiced include, but are not limited to: cleaning the external auditory canal with a solution designed to remove organic components, washing or rinsing the external auditory canal, applying external energy to the bony growths, imaging the target bony growths site, and the like. Such additional steps are known to one of skill in the art.

Utility

The subject methods find use in the treatment of conditions characterized by the presence of inorganic structures in the external auditory canal. As such, the subject methods and devices find use in the treatment of diffuse exostosis, tympanic sclerosis, and the like. Treatment by the subject methods includes inhibition of growth of the target inorganic structures, as well as growth prevention in the external auditory canal, as described above. Preferably, treatment by the subject methods results in the decrease in size of an inorganic structure. Most preferably, the inorganic structures are eliminated as a result of treatment by the subject methods.

Diffuse exostosis may be treated by application of the subject methods. Where the external auditory canal is partially or completely occluded by bony growths, the subject methods are used to flush a sufficient amount of an inorganic matter dissolution solution into the canal, thereby at least preventing further growth of the inorganic matter. Preferably, the inorganic matter dissolution solution applied will reduce the size of the bony growths, and even more preferably, will remove the inorganic structures entirely from the external auditory canal.

The subject methods also find use in the treatment of tympanic sclerosis. The thickening of the eardrum that is the result of inorganic matter present in the external auditory canal may be treated by the subject methods. The inorganic matter dissolution solution is flushed into the external auditory canal in an amount sufficient to prevent at least further growth of the inorganic matter. Preferably, the inorganic matter is reduced in size, and even more preferably, is removed completely from the external auditory canal.

The subject methods may be used in conjunction with other methods, including alcohol lavage, as part of a total treatment protocol.

A variety of hosts are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), lagomorpha (e.g., rabbits) and primates (e.g., humans, chimpanzees, and monkeys). In many embodiments, the hosts will be humans.

Devices

Also provided are devices for use in practicing the subject methods. The subject devices may be manual or automated devices, and may be simple or complex. An example of a simple, manual device is an ear dropper, e.g., a bottle filled with a fluid dispenser which can be placed into at least the opening of the external auditory canal, where the dropper is filled with the inorganic matter dissolution solution. In using this device, the operator may squeeze or otherwise dispense fluid from the bottle into the external auditory canal.

A more complex device includes an automated flushing device, which device typically includes a fluid introduction element and a fluid removal element. The fluid introduction element and fluid removal element may are typically dimensioned so as to be able to deliver fluid to, and remove fluid from, the external auditory canal, respectively. The device of this embodiment may take on a variety of configurations, including a tubular delivery structure made up of two lumens, one for fluid delivery and one for fluid removal, two tubular structures bound together, etc. The subject device may further include a reservoir for the dissolution solution and a second reservoir for storing aspirated fluid. The subject device may also include a pressurization element, to deliver the solution at a higher than normal pressure; a temperature modulation element, e.g., for warming the solution prior to flushing the external auditory canal; a aspiration element for providing negative pressure to the fluid removal element, and the like.

Systems

Also provided by the subject invention are systems for practicing the subject methods, e.g., for flushing the external auditory canal with an inorganic matter dissolution solution. The subject system typically include a device for administering the inorganic matter dissolution solution. The systems may further include a number of optional components, for example, pressure modulation elements, temperature modulation elements, fluid reservoirs, aspiration elements, and the like.

Kits

Also provided by the subject invention are kits for use in inhibiting the growth of the target inorganic structures in the external auditory canal. The subject kits at least include: an acidic solution or precursors thereof and a fluid delivery means for delivering the acid solution to the target structures. The solution or one or more precursors thereof may be present in a container(s), e.g. a flexible bag, a rigid bottle, etc. For kits that are to be used in methodologies in which the fluid is flushed through the external auditory canal, the amount of acidic fluid present in the kit ranges from about 0.5 to 500 liters, usually from about 0.5 to 200 liters and more usually from about 0.5 to 100 liters. In many of these embodiments, the amount of acidic fluid in the kit ranges from 0.5 to 5 liters, usually from about 0.5 to 2.0 liters and more usually from about 0.5 to 1.5 liters. In contrast, where the fluid is to be contacted statically with the target inorganic structure, the kit may include much less dissolution fluid. In these embodiments, the amount of dissolution fluid that may be present in the kit may range from about 0.5 ml to 100 ml, usually from about 5 ml to about 50 or 20 ml, and more usually from about 5 to 15 ml. As mentioned above, the kit may alternatively include precursors of the dissolution solution for use in preparing the solution at the time of use. For example, the precursors may be provided in dry form for mixing with a fluid, e.g. water, at the time of use. The kits also include a fluid delivery means for delivering the acidic fluid to the external auditory canal, such as the devices described above. The kits may further include one or more additional components and accessories for use with the subject fluid delivery/removal systems, including tubing for connecting the various fluid delivery components with fluid reservoirs, syringes, pumping means, etc., connectors, vacuum regulators, etc. Finally, the kits include instructions for practicing the subject methods, where such instructions may be present on one or more of the kit components, the kit packaging and/or a kit package insert.

The following example is provided by way of illustration and not by way of limitation.

Experimental

I. Dissolution of Bolus of Dahllite in 0.05N HCl with Various Ionic Strengths Using Pump at 69 ml/min

1. Introduction

Six dissolution experiments were conducted to determine the affect of ionic strength on the dissolution rate of carbonated hydroxyapatite in HCl.

2. Experimental

A Cole-Parmer peristaltic pump (model #7520-35) was used to deliver the demineralizing 0.05N HCl solution with varying NaCl concentrations to the sample of carbonated hydroxyapatite (i.e. dahllite), Ca_{8.8}(HPO₄)_{0.7}(PO₄)_{4.5}(CO₃)_{0.7}(OH)_{1.3}, in the form of a spherical bolus. In each case, a 100 3 mg bolus (dry weight) of carbonated hydroxyapatite was soaked in deionized water until there was no further weight gain. This weight was taken to be the initial weight of the bolus. The bolus was then transferred to a 12 ml disposable liquid transfer pipette and a peristaltic pump with a rubber stopper on one end of the tubing was attached. Solutions were pumped through the pipette past the bolus at a rate of approximately 69 ml/min in 5 minute time intervals and the weight of the bolus was measured at the end of each interval. The dissolution process was continued until the weight of the bolus was less than 5 mg. The NaCl concentrations used were: 0, 5.8 (isotonic), 11.6, and 25 g/L.

3. Results

The results of the six dissolution experiments are tabulated below. A table of the respective half-lives follows. The wet weight of the bolus at t=0 is represented by m(o), and m(t) is the weight at a given time interval (m=mass).

TABLE 1
Dissolution of Bolus of 0.05 N HCl with Various Ionic Strengths

log[m(t)/m(0)]

| Time | | No | 5.8 g | 5.8 g | 11.6 g | |
|---|---|---|---|---|---|---|
| (min) | No salt | salt (2) | NaCl | NaCl (2) | NaCl | 25 g NaCl |
| 0.0 | 0.0000 | 0.0000 | 0.0000 | 0.0000 | 0.0000 | 0.0000 |
| 5.0 | −0.0569 | −0.0982 | −0.0789 | −0.0822 | −0.1209 | −0.1300 |
| 10.0 | −0.1374 | −0.2121 | −0.1926 | −0.1803 | −0.2287 | −0.3014 |
| 15.0 | −0.2403 | −0.3212 | −0.3322 | −0.2906 | −0.3973 | −0.5792 |
| 20.0 | −0.3594 | −0.4491 | −0.5195 | −0.4514 | −0.6717 | |
| 25.0 | −0.4765 | −0.5907 | −0.8683 | −0.6736 | | |
| 30.0 | −0.6273 | −0.7788 | | −1.2653 | | |
| 35.0 | −0.8154 | −1.0740 | | | | |

Half-lives for the Dissolution of Bolus in 0.05 N HCl

| | | | | | | |
|---|---|---|---|---|---|---|
| Half-life | | No | 5.8 g | 5.8 g NaCl | 11.6 g | |
| (min) | No Salt | Salt (2) | NaCl | (2) | NaCl | 25 g NaCl |

| | | | | | |
|---|---|---|---|---|---|
| 15.8 | 12.7 | 11.6 | 11.8 | 10.5 | 8.8 |

4. Discussion and Conclusion

The half-life data and log[m(t)/m(0)] vs. time show that increasing the ionic strength of the solution increases the dissolution rate. This contradicts the Kinetic Salt Effect theory which says that increasing the ionic strength of a solution decreases the reaction rate between oppositely charged ions due to a decrease in electrostatic attraction between the ions. In this case, Na⁺ and Cl⁻ ions should theoretically decrease the electrostatic attraction between H⁺ and both HPO4²⁻ and PO4³⁻ and slow the rate of dissolution.

II. Demineralization of a Tympanic Membrane

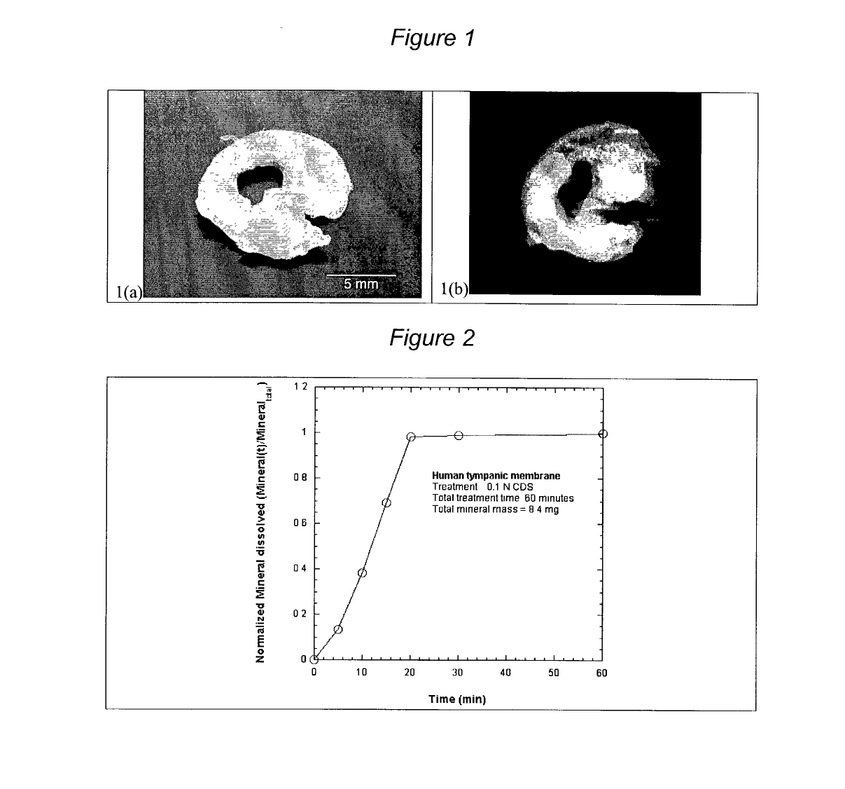

The use of a demineralization technology to remove the mineralized deposits from the tympanic membrane has been evaluated. A calcified tympanic membrane was retreived post-surgical excision. The excised tissue appeared intact with a perforation just off center in the sample. See FIG. 1a. High resolution contact radiographs confirmed the presence a large mineral mass. See FIG. 1b. Visual and tactile examination of the sample demonstrated that the mineral mass within the tympanic membrane was completely covered with a thin fibrous tissue layer. The tympanic membrane sample was pre-treated with a saline lavage (0.9% sodium chloride) for 3-two minute treatment periods. The sample was then treated for a period of 20 minutes by submerging the tissue in 50 ml of a 0.05N HCl solution (CDS). During the treatment period, the sample container was placed into an ultrasonic bath (42 kHz Branson 5510 Ultrasonic Cleaner, Danbury, Conn.) to provide mixing of the solution. At 4-five minute intervals, solution samples were removed for mineral analysis using atomic absorption spectroscopy (AAS). Post-CDS treatment, the tissue was treated with 25 ml of buffer to neutralize the CDS solution for a period of 20 minutes, followed by a 2 minute lavage of the tissue with saline. Following treatment, the tissue remained intact and appeared to be more supple.

To determine the level of demineralization of the tissue, the tissue was treated with CDS until all mineral was dissolved and the total mineral in the tissue calculated (Mineralₜₒₜₐₗ). The dissolution behavior of a calcific human tympanic membrane treated with CDS is plotted as a function of time in FIG. 2. The normalized mineral mass (Mineral(t)/Mineralₜₒₜₐₗ), mineral mass at each time point relative to the total mineral mass within the sample, is plotted as a function of time. Note that a steady dissolution of the mineral occurs with exposure to CDS over the 20 minute treatment. It is observed that over this 20 minute treatment approximately 98% of the mineral deposits within the tissue have been removed. These results demonstrate the feasibility of dissolving mineral from tympanic membrane tissue using CDS solution.

It is evident that the subject invention provides an important new way to treat conditions characterized by the presence of inorganic structures in the external auditory canal. Benefits of the subject invention include a non-surgical procedure that is less traumatic, e.g., poses no risk of further damage or hearing loss to the external ear canal, and therefore provides significant advantages over the current surgical procedures. As such, the subject invention represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of at least inhibiting growth of an inorganic structure present in an external auditory canal of a host, said method comprising:
contacting said structure with an effective amount of an inorganic matter dissolution solution to prevent growth of said structure.

2. The method according to claim 1, wherein said contacting is achieved by flushing said external auditory canal with said inorganic matter dissolution solution.

3. The method according to claim 1, wherein said inorganic matter dissolution solution is an acidic solution.

4. The method according to claim 3, wherein said acidic solution has a pH that does not exceed about 4.

5. The method according to claim 4, wherein said acidic solution is a hydrochloric acid solution.

6. A method for reducing the size of an inorganic structure present in the external auditory canal of a host, said method comprising:
flushing said external auditory canal with an effective amount of an inorganic matter dissolution solution to reduce the size of said structure.

7. The method according to claim 6, wherein said inorganic matter dissolution solution is an acidic solution.

8. The method according to claim 7, wherein said acidic solution has a pH that does not exceed about 4.

9. The method according to claim 8, wherein said acidic solution is a hydrochloric acid solution.

10. A method for removing an inorganic structure present in the external auditory canal of a host, said method comprising:
flushing said external auditory canal with an effective amount of an inorganic matter dissolution solution to remove said inorganic structure.

11. The method according to claim 10, wherein said inorganic matter dissolution solution is an acidic solution.

12. The method according to claim 11, wherein said acidic solution has a pH that does not exceed about 4.

13. The method according to claim 12, wherein said acidic solution is a hydrochloric acid solution.

14. A method for preventing the formation of an inorganic structure in an external auditory ear canal site, said method comprising:
flushing said external auditory canal with a sufficient amount of an inorganic matter dissolution solution to prevent formation of an inorganic structure in said external auditory canal.

15. The method according to claim 14, wherein said inorganic matter dissolution solution is an acidic solution.

16. The method according to claim 15, wherein said acidic solution has a pH that does not exceed about 4.

17. The method according to claim 16, wherein said acidic solution is a hydrochloric acid solution.

18. A method for treating diffuse exostosis in a host, said method comprising:
flushing an external auditory canal of said host with an effective amount of an inorganic matter dissolution solution to at least treat said diffuse exostosis.

19. The method according to claim 18, wherein said inorganic matter dissolution solution is an acidic solution.

20. The method according to claim 19, wherein said acidic solution has a pH that does not exceed about 4.

21. The method according to claim 20, wherein said acidic solution is a hydrochloric acid solution.

22. A device for delivering an inorganic matter dissolution solution to an external auditory canal of a host, said device comprising:
a flushing element for flushing an external auditory canal site with an inorganic matter dissolution solution.

23. A system for delivering an inorganic matter dissolution solution to an external auditory canal, said system comprising:
a device according to claim 22; and
an inorganic matter dissolution solution.

24. A kit for use in delivering an inorganic matter dissolution solution to an external auditory canal, said kit comprising at least one of:
a device according to claim 22; and
an inorganic matter dissolution solution or precursors thereof.
